(19) United States
(12) Patent Application Publication
(10) Pub. No.: US 2016/0262876 A1
(43) Pub. Date: Sep. 15, 2016

(54) Intraocular Lens with Enhanced Depth of Focus and Reduced Aberration

(71) Applicants: Charles DeBoer, Sierra Madre, CA (US); Mark Humayun, Glendale, CA (US); Yu-Chong Tai, Pasadena, CA (US)

(72) Inventors: Charles DeBoer, Sierra Madre, CA (US); Mark Humayun, Glendale, CA (US); Yu-Chong Tai, Pasadena, CA (US)

(21) Appl. No.: 15/065,380

(22) Filed: Mar. 9, 2016

Related U.S. Application Data
(60) Provisional application No. 62/130,277, filed on Mar. 9, 2015.

(51) Int. Cl. A61F 2/16

(52) U.S. Cl. CPC A61F 2/1659; A61F 2002/1699; A61F 2250/0004

(57) ABSTRACT

Embodiments of intraocular lenses described herein include features that enhance depth of focus and/or reduce chromatic aberration. These features may include different optical fluids, multiplexed or asymmetric lens arrangement, dispersive or diffusive elements, and others.

### CROSS-REFERENCE TO RELATED APPLICATION

This application claims priority to, and the benefits of, U.S. Ser. No. 62/130,277, filed on Mar. 9, 2015, the entire disclosure of which is hereby incorporated by reference.

### FIELD OF THE INVENTION

In various embodiments, the present invention relates generally to an implantable intraocular lenses and, more specifically, to fluid-filled intraocular lenses.

### BACKGROUND

The crystalline lens of the human eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and obstruct vision.

Conventionally, cataract treatments involve surgically removing the opaque lens matrix from the lens capsule using, for example, phacoemulsification and/or a femtosecond laser through a small incision in the periphery of the patient's cornea. An artificial intraocular lens (IOL) can then be implanted in the lens capsule bag—the sack-like structure remaining within the eye following extracapsular cataract extraction; the lens “capsule” is the thin clear membrane that surrounds the natural crystalline lens—to replace the natural lens. Generally, IOLs are made of a foldable material, such as silicone or uncrosslinked acrylics, to minimize the incision size and required stitches and, as a result, the patient's recovery time. IOLs are described, for example, in U.S. Pat. Nos. 8,771,347 and 8,715,345 and U.S. Patent Publ. 2013/0317607, the entire disclosures of which are hereby incorporated by reference.

An IOL should provide good visual acuity at far, intermediate and near distances. Several approaches have been used to achieve this objective, including bifocal lenses, multifocal lenses that project simultaneous near- and far-focus images on the retina, and accommodating intraocular lenses (AIOLs), which interact with the natural musculature of the eye to adjust focal length.

Visual acuity also requires that corrective lenses exhibit low chromatic aberration—i.e., the tendency of a lens to focus different wavelengths of light at different distances, with the result that the various perceivable colors are not all in focus at the same plane. This is experienced as fringes of color along boundaries between light and dark. Scientifically, the Abbe number is used to describe the dispersion of a material. Higher Abbe numbers correspond to lower levels of dispersion, and chromatic aberration is inversely proportional (or inversely related) to the Abbe value. This means that the chromatic aberration of a lens increases as the Abbe value decreases, and vice versa. In general, high-index materials have lower Abbe values than conventional plastic and crown glass lens materials, which makes the former more likely to produce symptoms of chromatic aberration. The higher the refractive index of the material, the lower the Abbe value is likely to be.

Achieving depth of focus over broad distance regions and minimizing chromatic aberration represent separate challenges that are often interrelated, since measures taken to increase one form of acuity can adversely affect the other.

### SUMMARY

Embodiments of the present invention include features that enhance depth of focus, reduce chromatic aberration, or do both.

Accordingly, in a first aspect, the invention pertains to an intraocular lens comprising a membrane defining a central chamber for containing an optical fluid and, when filled, to provide vision correction when implanted in a patient's eye; the membrane has an optical axis and opposed anterior and posterior optical surfaces perpendicular to the optical axis. In various embodiments, a first portion of at least one of the optical surfaces has a higher optical density than a second portion thereof different from the first portion, such that more light passes through the second portion than the first portion. For example, the second portion may have a circular shape (and a diameter oif, e.g., 3 mm) and be centrally located on the corresponding optical surface. In some embodiments, the first portion is a peripheral portion surrounding the second portion. The first portion may have an absorbance of at least 70% for visible light, and in some embodiments, of at least 95% for visible light.

The peripheral portion of higher optical density may be on the anterior surface, the posterior surface, or both surfaces. In various embodiments, the peripheral portion comprises a plurality of concentric zones of differing optical densities. The concentric zones may increase in optical density with radial distance from the central zone. In some embodiments, the peripheral portion increases continuously in optical density from the central portion. The increase may be linear, nonlinear, or stepwise. In other embodiments, the second portion is a pattern of apertures distributed over corresponding optical surface.

At least one of the anterior and posterior optical surfaces may comprise a first, central lens portion having a first optical power; and a second lens portion at least partially surrounding the first lens portion, the second lens portion having a second optical power different from the first optical power. For example, the second lens portion may be a peripheral (e.g., annular) portion discrete from and fully surrounding the first lens portion. In some embodiments, the lens has a third lens portion, and the second and third lens portions are discrete, have different lens powers, and collectively surround the first lens portion.

In some embodiments, at least one of the optical surfaces and/or the optical fluid has an Abbe number different from at least one other of the optical surfaces and/or the optical fluid. Some embodiments also feature a diffractive element on at least one of the anterior or posterior optical surfaces.

In another aspect, the inventio relates to an intraocular lens comprising a membrane defining a central chamber for containing an optical fluid and, when filled, to provide vision correction when implanted in a patient's eye; the membrane has an optical axis and opposed anterior and posterior optical surfaces perpendicular to the optical axis. In various embodiments, the intraocular lens comprises a first, central lens portion having a first optical power; and a second lens portion at least partially surrounding the first lens portion, the second lens portion having a second optical power different from the first optical power.

In some embodiments, the second lens portion is a peripheral portion discrete from and fully surrounding the first lens portion; for example, the second lens portion may be annular. In various implementations, the lens comprises a third lens portion, and the second and third lens portions are discrete, have different lens powers, and collectively surround the first lens portion. For example, the first and second lens portions may be arranged to allow for different proportions of near and far focus based on a patient's pupil dilation. Lens power may vary continuously along a radius of the intraocular lens through the first and second lens portions.

In some embodiments, the lens includes a diffractive element. The first and second lens portions may provide distance focus over substantially non-overlapping distance regions of focus. The first and second lens portions may be fabricated as a multifocal lens, and the the overall intraocular lens may be an accommodative intraocular lens.

In still another aspect, the invention relates to an intraocular lens comprising an envelope membrane defining first and second opposed lens elements and a middle portion therebetween. In various embodiments, the first and second lens elements have Abbe numbers different from each other and/or from the middle portion.

In some embodiments, the first and second lens elements share a common surface internal to the intraocular lens. The envelope membrane may define an interior and the lens may further include, in the interior, a substantially transparent internal membrane defining first and second internal chambers on opposed sides of the membrane, each for containing an optical fluid; the membrane is perpendicular to an optical axis of the intraocular lens, and the first and second internal chambers are filled, respectively, with first and second optical fluids having different Abbe numbers. The envelope membrane may have opposed anterior and posterior surfaces on opposite sides of the spanning membrane. Each of the internal chambers and the surface associated therewith may behave as separate first and second lens elements each having an associated focal length, the intraocular lens conforming to the relationship f₁×V₁+f₂×V₂=0, where f₁ and f₂ are the focal lengths of the first and second lens elements and V₁ and V₂ are the Abbe numbers of the first and second optical fluids. The first and second lens elements may have different refractive powers. In some embodiments, one of the lens elements is solid and the other lens element is a fluid-filled chamber. The intraocular lens of claim 31, wherein the Abbe numbers of the first and second lens elements range from 10 to 100. Portions of the lens may, in various embodiments, have varying Abbe numbers; for example, the Abbe numbers may vary in a gradient. The internal chambers may be symmetric or asymmetric. Either or both lens elements and/or the middle portion may be doped with a dopant (e.g., a metal oxide).

Yet another aspect of the invention relates to an intraocular lens comprising, in various embodiments, an envelope membrane defining an outer surface and an interior and a lens element suspended within the interior, wherein the envelope membrane has a curvature defining a first refractive power and the interior lens element having a second refractive power different from the first refractive power. In some embodiments, the interior is filled with an optical fluid. The intraocular lens may conform to the relationship f₁×V₁+f₂×V₂=0, where f₁ and f₂ correspond to the first and second refractive powers and V₁ and V₂ are the Abbe numbers of the optical fluid and the interior lens element, respectively. The Abbe numbers may range from 20 to 100. In some embodiments, the interior lens element is solid.

In still another aspect, the invention pertains to an intraocular lens comprising an envelope membrane defining an outer surface having anterior and posterior sides and an interior. In various embodiments, the lens has a diffractive element on at least one of the anterior or posterior sides, the envelope membrane has a curvature defining a refractive power of the lens and the diffractive element reduces chromatic aberration thereof. The intraocular interior may be filled with an optical fluid.

Still another aspect of the invention relates to an intraocular lens (e.g., an accommodative intraocular lens) comprising a membrane defining a central chamber for containing an optical fluid and, when filled, to provide vision correction when implanted in a patient's eye; the membrane has an optical axis and opposed anterior and posterior optical surfaces perpendicular to the optical axis. In various embodiments, one or more of the optical surfaces has a radially symmetric asphericity to improve depth of focus. The spherical aberration may be between −0.05 and −0.4 μm. The spherical aberration may vary with accommodative distance or with radial distance from the optical axis.

The term “substantially” or “approximately” means ±10% (e.g., by weight or by volume), and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 is a sectional view of a conventional fluid-filled IOL.

FIG. 2 is a front elevation of an IOL modified for extended depth of focus in accordance with an embodiment of the invention.

FIG. 3 is a front elevation of a variation of the embodiment shown in FIG. 2, where the opaque region varies radially in density.

FIG. 4 is a front elevation of a variation of the embodiment shown in FIG. 2, where multiple small apertures are used instead of a single aperture.

FIG. 5A and 5B are side schematic elevations of an IOL that achieves increased depth of focus using multiple optical zones.

FIG. 6A is a front elevation of an intraocular lens having a series of curvilinear lens portions distributed around a central lens portion.

FIG. 6B is a side elevation of the lens shown in FIG. 6A and also including a diffractive element.

FIGS. 7 and 8 graphically depict the effect of overlapping focus regions for IOLs with “multiplexed” lens elements.

FIG. 9 is a side elevation of an intraocular lens having materials with different dispersive properties.

FIG. 10 is a side elevation of an intraocular lens arranged as an optical doublet with reduced chromatic aberration.

FIG. 11 shows a variation of the lens depicted in FIG. 10, this embodiment having asymmetric lens elements.

FIG. 12 is a side elevation of an intraocular lens including a fully internal secondary lens element.

FIG. 13 is a side elevation of an intraocular lens including a diffractive optic.

### DETAILED DESCRIPTION

### Enhanced Depth of Focus

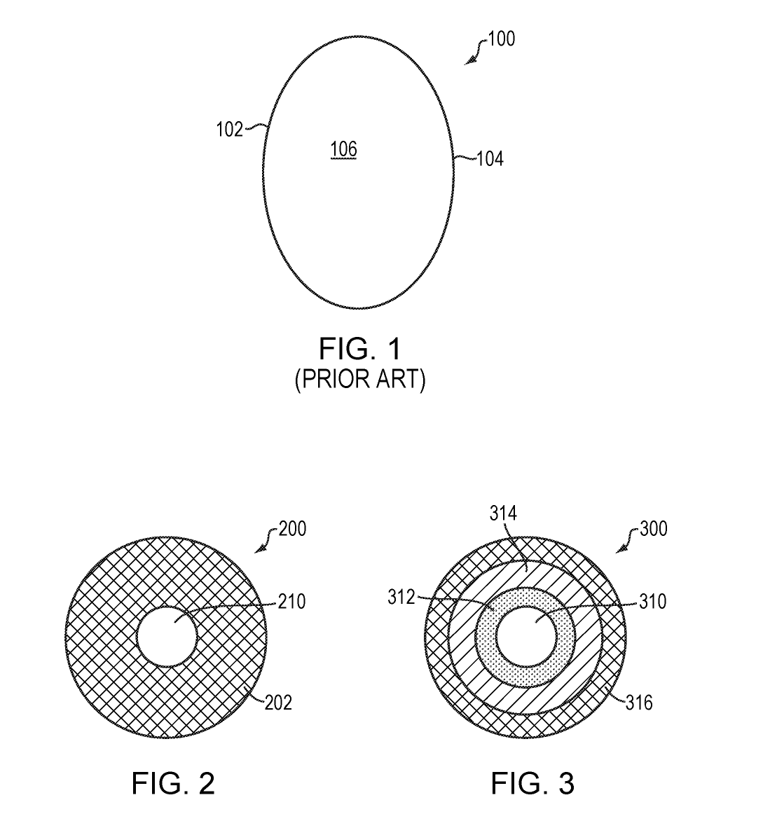

FIG. 1 illustrates a conventional fluid-filled IOL 100, which has an anterior surface 102 and a posterior surface 104. A filling fluid (e.g., silicone oil) occupies the interior 106 of the IOL 100. The fluid-filled lens 100 need not be a static lens; in certain embodiments it is an AIOL, in which case the enhancements to depth of focus (DOF) described herein supplement accommodation from the lens 100 itself

As an example, if the IOL 100 has +4 diopters of accommodative capability, and an extended DOF corresponding to +2 diopters, the total visual acuity can be maintained over a range of six diopters. This is two diopters more than an accommodating intraocular lens with four diopters of accommodation, and four diopters more than a two-diopter extended DOF lens. In this manner, visual outcomes are improved by using both techniques to increase visual acuity.

FIG. 2 illustrates an IOL 200 in which part of the lens has been occluded. In particular, the anterior surface 202 (e.g., the entire anterior half of the lens surface) is made opaque (e.g., using a dye or a conforming polymeric mask) or at least reduced in transmissivity except for an optical aperture 210 through which light can pass. (The aperture 210 is not a physical aperture through the anterior surface 202, but is instead an optical aperture of substantially complete transmissivity for visible light.) This limits the directions from which light entering the lens 200 may originate; the dominant light rays that pass through the lens 200 are paraxial. This increases the lens DOF at the price of reduced light collection. In other embodiments, the aperture is located on the posterior side of the lens 200, which side is otherwise opaque, or the entire lens 200 except for opposed apertures on the anterior and posterior faces may be opaque. In various embodiments, the aperture is a small circular area (e.g., 3 mm diameter) of increased optical transmission with a surrounding area of opacity, but may take other shapes (e.g., elliptical).

The optical density of the opaque region can vary, although a typical range, expressed as absorbance, is 50% to 100% with respect to visible light, and most typically 70% or 80% to 100%. The IOL 300 shown in FIG. 3 has a central optical aperture 310 with substantially complete (e.g., 100%) transmission and a plurality of concentric annuli 312, 314, 316 that have decreasing degrees of visible light transmission. In this manner, discrete steps of light attenuation are used to create a favorable light profile along with increased DOF due to the aperture effect. Light rays that are not close to the visual axis are progressively attenuated by the concentric rings 312, 314, 316. For example, the clear aperture 310 may occupy the central 2.5 mm of the lens, the first concentric annulus 312 may extend to 3.5 mm with 50% transmission, the second annulus to 5.5 mm with 25% transmission, and finally no transmission beyond 5.5 mm. It should be noted that while the head-on view of FIG. 3 shows the zones 312, 314, 316 as perfect annuli, the curvature of the anterior or posterior surface of the lens means that the zones will actually be stretched somewhat to present the annuli as illustrated and experienced by the patient.

The degree of attenuation with radial distance from the aperture 310 need not be stepped; it may be continuous, and it need not be linear. For example, a gradient of radially varying transmissivity may follow a custom profile or may be a smooth taper from one level of transmission to another (e.g., 90% transmission at the periphery of the aperture 310 in the center to 40% at the periphery of the lens anterior (or posterior) itself). An attenuation gradient maintains the DOF improvement as the patient's pupil dilates, but permits light collection when it is most needed. That is, a balance may be struck between overall vision and DOF improvement in low-light conditions, since DOF improvement is irrelevant if the patient cannot see; it is when the patient's pupil is fully dilated that the need for light collection is greatest, and this is provided by the radially decreasing opacity outside the aperture 310—particularly since circular area increases with the square of the radius.

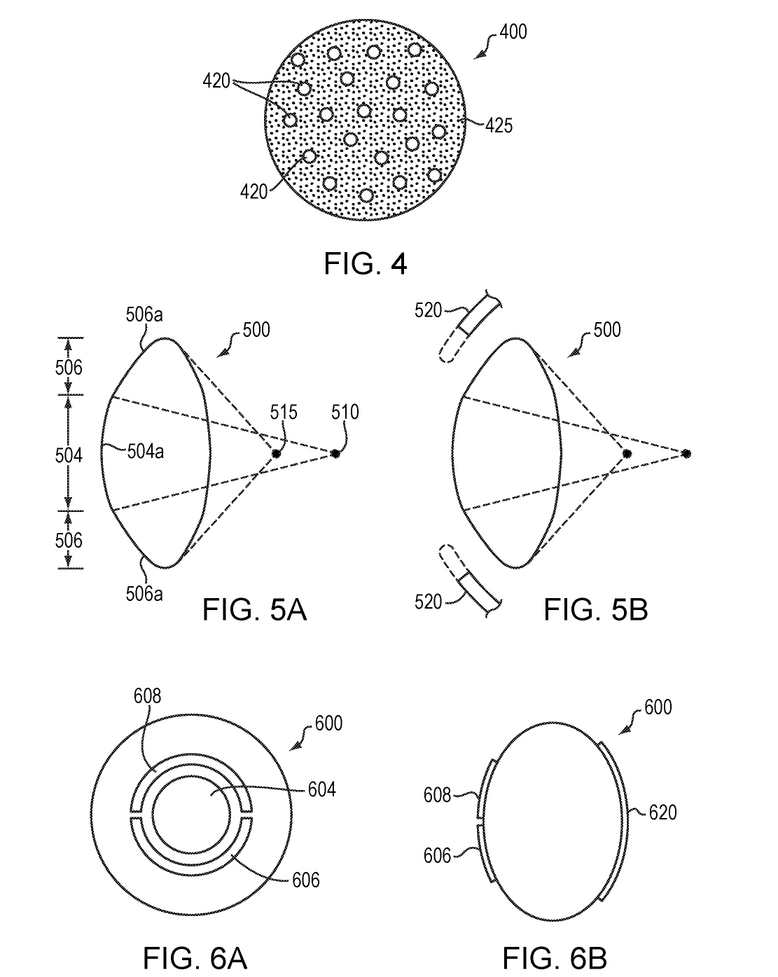

The opacity pattern not only need not vary linearly, but also need not vary radially. With reference to FIG. 4, an IOL 400 is provided with a pattern of clear optical apertures 420 that are substantially transparent to visible light, and distributed over a background region 425 that is opaque or exhibits reduced optical density relative to the apertures 420. The apertures 420 act collectively with an effect similar to a pin-hole camera and therefore increase the DOF of the lens 400. Within limits, increasing the number of pin holes 420 (with diameter between 500 μm and 2 mm) increases the total light transmitted through the lens without substantially detracting from the DOF improvement afforded by the pin holes. To maximize the entry of light while minimizing reduction of DOF, the pin holes 420 may be substantially uniformly spaced apart. If the pattern becomes too dense, diffraction will defeat the pin-hole effect and the DOF benefits will degrade. The same is true if the pin holes are too large relative to their spacing from each other.

In other embodiments, the lens utilizes aberration to increase DOF. For example, within limits, spherical aberration increases DOF. This is because the light passing through different parts of the pupil may focus at different distances from the cornea, therefore being perceived as a non-sharp image in the paraxial and peripheral regions of the pupil. Consequently, a spherical aberration on the anterior and/or posterior surfaces of the lens increases DOF. Although varying by individual, a spherical aberration range between −0.05 to −0.4 μm, implemented as a radially symmetric asphericity, will improve DOF. For an accommodating fluid-filled IOL, the spherical aberration range may be implemented as a function of accommodation (e.g., at near infinity, the spherical aberration is 0.1, at 2 m the spherical aberration is 0.3). The relationship between distance and aberration may not be linear. By implementing an accommodation-varying spherical aberration into the IOL, the change per unit distance in the modulation transfer function may be controlled, thereby allowing for an improved balance of DOF and optical clarity throughout the full range of vision. The spherical aberration may also vary by radial distance from the aperture within the optical zone (e.g., as concentric annuli 312, 314, 316) to balance the DOF of images in the central and peripheral regions.

Another approach to increasing DOF is to employ a lens with multiple optical zones, each having a different focal length. With reference to FIG. 5A, the IOL 500 has a first, central optical zone 504 occupying the central area 504a of the lens 500, and a second, peripheral optical zone 506 occupying the peripheral area 506a of the lens 500. The central optical zone 504 has a long focal length that focuses light at the point 510, and the peripheral optical zone 506 has a short focal length that focuses light at the point 515. Thus, the center of the lens 500 has higher power and corrects focus for near distances, while the periphery of the lens 500 has lower power and corrects focus for farther distances. As shown in FIG. 5B, with the patient's pupil 520 constricted, light passes primarily through the central optical zone 504 (for near distances). In certain embodiments, there is a difference between the near and far power of less than 10 diopters. In other embodiments the difference between the near and far power is less than 4 diopters. In yet other embodiments the difference between near and far power is less than 2 diopters.

When the patient's pupil 520 is dilated, light passes through both optical zones 504, 506. It should be stressed, however, that there may be more than two lens regions of differing power depending on the application. These regions may be discrete and annular as shown in FIG. 5A, or they may be distributed randomly or in a configuration other than annular. In general, the lens power is distributed in a manner that allows for different proportions of near and far focus based on pupil dilation. With annular lens regions of differing power, these proportions are discrete and the effect is stepped. In other embodiments, the lens power varies continuously and gradually along the radius of the lens 500. In still other embodiments, as shown in FIG. 6A, the lens 600 has a central zone 604 analogous to the zone 504 described above, but additional curvilinear zones 606, 608 of differing power. As illustrated, these zones 606, 608 may collectively form an annular region, but this is not necessary. This lens “multiplexing” may be achieved, for example, by using either differing radii of curvature along the surface or by using diffractive lens elements. Alternatively, the anterior surface of an IOL may have a series of diffractive lens elements that allow focusing on two or more focal planes. In other approaches, a plurality of diffractive rings are added to the optical surface, or a nonbinary profile of rings is added on top of the aperture lens to generate unbalanced optical path differences across the lens face.

Although only far and near vision were discussed above for simplicity, it is to be understood that areas of the lens 500, 600 may also correspond to intermediate viewing distances. This is advantageous in that when focused at intermediate distances, the natural DOF of the eye, including the pinhole effect of the pupil, will allow a range of focal lengths to be in focus around that intermediate distance. Therefore, an intermediate focal power will improve DOF over a range of distances. (The terms “power” and “focal length” are herein used interchangeably.)

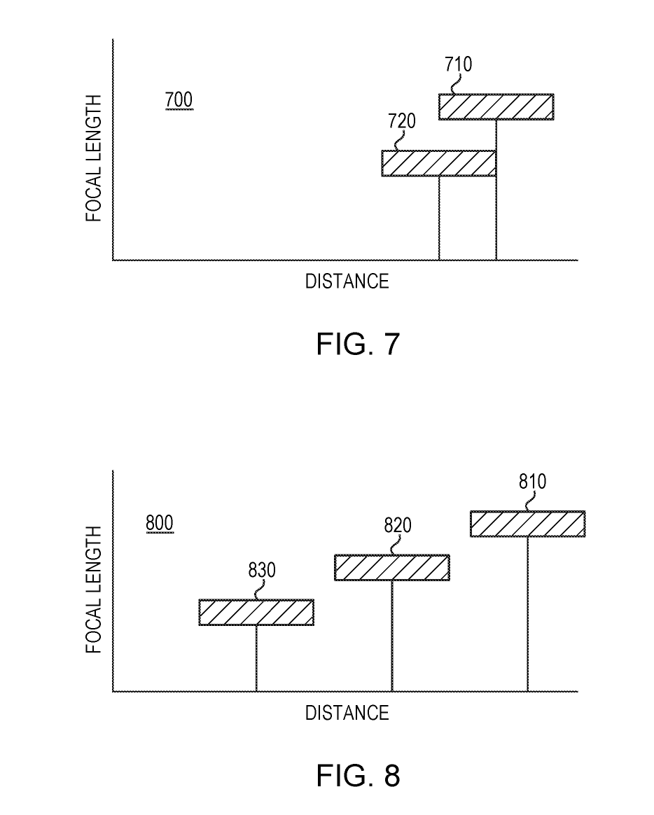

FIGS. 7 and 8 illustrate how this concept can be used to optimize the focal lengths of a multi-zone lens. The graph 700 shows the area of focus of two lenses. The first lens provides vision correction over a distance region 710, while the second lens provides vision correction over an overlapping distance region 720. Although the center of each region 710, 720 corresponds to the sharpest point in the image, the distance region of focus—where the patient sees with 20/20 vision—extends over the distances indicated at 710, 720. Because the focused distance regions 710, 720 overlap, however, the overall distance within focus is less than the sum of the distances covered by each of the regions 710, 720—i.e., it is less than optimal. FIG. 8 depicts the focus regions 810, 820, 830 of a lens with three different focal lengths. The distance region 810 corresponds to far intermediate vision, which allows far vision as well as a large range of intermediate vision to be in focus. The distance region 820 is fully intermediate vision. The distance region 830 centers at an intermediate distance, which allows near vision to be in focus along with a range of vision up to the intermediate distance region 820. In this manner, full depth of field can be captured using multiple focal lengths within a single lens. The three lens regions may correspond to the optical zones 604, 606, 608 of the lens 600 shown in FIG. 6A.

For example, the anterior portion, posterior portion and/or interior lens portion may be realized as a multifocal IOL, which may be manufactured using, for example, apodized, diffractive, and/or refractive optics. When the lens is an accommodating IOL, the accommodative effect of the lens allows for adjustable focus, while the multifocal portion of the lens increases DOF.

In certain embodiments, a multifocal lens element with a small add power is employed in conjunction with an accommodating IOL so that the combined lens components provide near, intermediate, and far vision. In preferred embodiment, the multifocal lens has an add power of +4 or less. In other embodiments the add power is +2 or less. When used in conjunction with an accommodating IOL, the 2 diopters of add from the multifocal lens element are added to the accommodative focusing power of the lens for a larger range of achievable lens powers. The lower add minimizes visual disturbance while the accommodating IOL provides enhanced DOF. Compared to a conventional rigid multi-focal IOL, the visual disturbances (e.g., halos and glare) will be reduced with an accommodating fluid-filled IOL as the added power at any given point has a minimal difference in absolute power compared with one or more accommodated multifocal portions.

As noted above, diffractive elements can be used to multiplex lenses within a single structure. If needed, a diffractive element 620 may be included on the opposite side of the lens (e.g., the posterior side if the diffractive lens elements are on the anterior side) to cancel aberration as shown in FIG. 6B. As is well known, diffractive elements are thin phase components that operate by means of interference and diffraction to alter the distribution of light passing therethrough. Other approaches for increasing DOF include interferometric techniques such as optical phase engraving to create a constructive interference along a focus channel.

### Managing Chromatic Aberration

To maximize visual acuity, chromatic aberration should be minimized. One complication of increasing DOF can be increased chromatic aberration induced by the lens. This leads to reduced visual quality of the image and dispersion of colors.

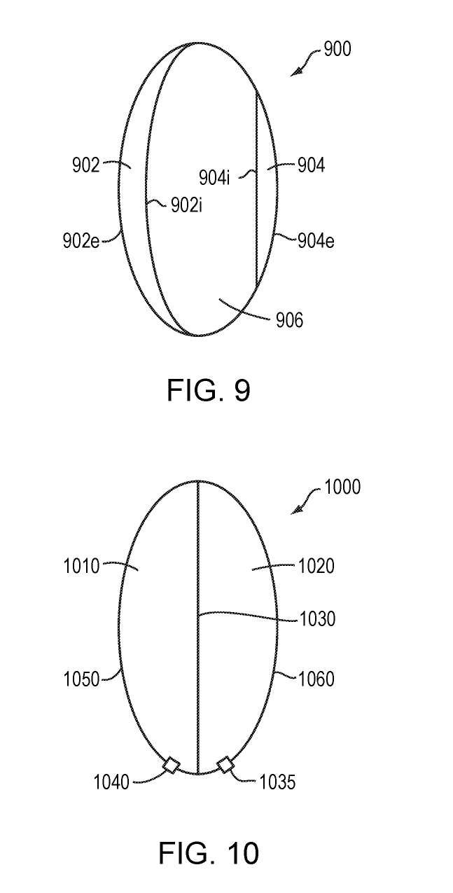

FIG. 9 shows an IOL 900 that includes an anterior lens portion 902, a middle portion 906 filled with an optical fluid as discussed above, and a posterior portion 904. The anterior portion 902 and, in various embodiments, the posterior portion 904 as well have optical power, and the central portion 906 contributes to optical performance as well. By using differing materials with different dispersive properties or Abbe numbers, it is possible to reduce overall chromatic aberration. For example, chromatic aberration induced by the anterior lens element 902 may be corrected by the central portion 906 or and/or by posterior portion 904. To generalize, different portions of the lens may produce different amounts of dispersion, and other portions of the lens may compensate for this. In certain embodiments, portions of the lens 900 or the entire lens is a gradient index lens. In other embodiments, portions of the lens have varying (e.g., gradient) Abbe numbers. As an example, the Abbe number of a material may be altered by doping (e.g., by nanocomposites or nanoparticles such as metal oxides (TiO₂ with Abbe number=14) and (ZrO₂ with Abbe number=32)). Dopants may be introduced into the lens material during manufacture.

The anterior portion 902 and the posterior portion 904 may also be separate chambers and each surface—external and internal anterior surfaces 902e, 902i and external and internal posterior surfaces 904e, 904i—may be doped separately. Alternatively or in addition, the optical fluid within one or more of the chambers 902, 904, 906 may be doped.

In other embodiments, the IOL has two or more separate compartments and behaves as a compound lens. For example, in optics, a “doublet” generally refers to two thin lenses in contact. As shown in FIG. 10, a doublet can be formed in an IOL 1000 by defining separate anterior and posterior compartments 1010, 1020 by a transparent membrane 1030. Each of the compartments 1010, 1020 has a separate fill valve 1035, 1040 to permit the compartments to be separately filled, e.g., with different optical fluids. In this way, the lens 1000 can be made to behave like an achromatic doublet lens, in which materials with differing dispersion (i.e., the different optical fluids) are used as components of the doublet. Furthermore, the anterior surface 1050 and the posterior surface 1060 can have different curvatures and, therefore, different refractive powers.

For a doublet consisting of two thin lenses in contact, the Abbe number of the lens materials is used to calculate the correct focal length of the lenses to ensure correction of chromatic aberration. If the focal lengths of the two lenses for light at the yellow Fraunhofer D-line (589.2 nm) are f₁ and f₂, then best correction occurs for the condition

f₁×V₁+f₂×V₂=0 (Eq. 1)

where V₁ and V₂ are the Abbe numbers of the materials of the first and second lenses, respectively. This demonstrates that one portion of the doublet lens 1000 must have a negative focal length for ideal correction.

Accordingly, for the embodiment depicted in FIG. 10, one of the chambers 1010, 1020 has a positive focal length, while the other chamber has a negative focal length. By choosing appropriate materials with different Abbe numbers so that Eq. 1 is satisfied, the overall chromatic aberration is reduced. Most straightforwardly, the chambers 1010, 1020 are filled with different optical fluids. Alternatively, one of the chambers 1010, 1020 may be replaced by a solid (e.g., crosslinked polymer) lens having an Abbe number different from that of the optical fluid in the remaining chamber. As an example, a filling fluid with a high Abbe number may be used to reduce the dispersion of light, thereby increasing the net Abbe number (regular optical glasses have an Abbe number between 25 and 70). For example, methanol has a low Abbe number of 13, whereas deionized water has a high Abbe number of 55. Suitable Abbe numbers for this and other embodiments range from 10 to 100.

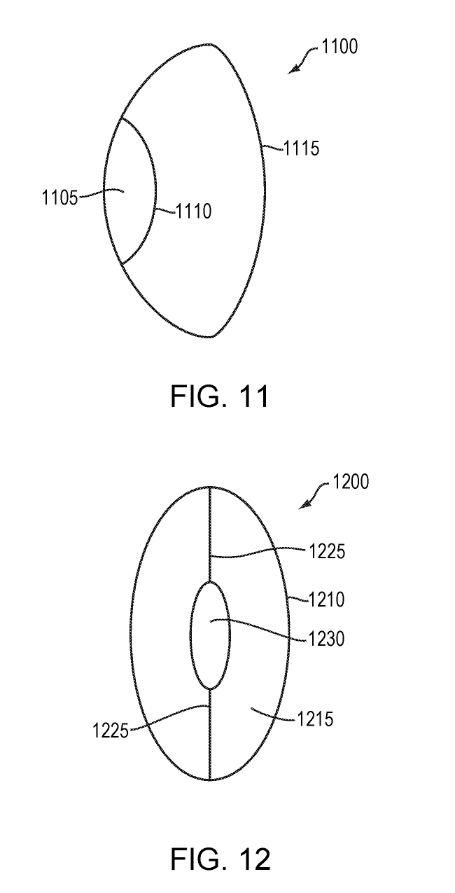

The chambers 1010, 1020 need not be symmetric as shown in FIG. 10. Instead, with reference to FIG. 11, an IOL 1100 may have an anterior lens element 1105 along or, in some cases, displaced from the overall optical axis of the IOL 1100. The lens element 1105 may be solid or defined by an interior membrane 1110 and filled with an optical fluid. The lens element 1105, as illustrated, has a positive power while the remainder of the IOL 1100 exhibits a negative power due to the curvature of the anterior surface 1115. In this case, different focal lengths, in lieu of or in addition to different materials, cause Eq. 1 to be satisfied.

In another variation, illustrated in FIG. 12, the IOL 1200 is defined by an outer polymeric envelope or shell 1210 that defines an interior chamber 1215, within which is mounted (e.g., mechanically held in place by struts 1225) an internal lens 1230. The lens 1230 has a negative focal power while the overall lens 1210 has a positive focal power. As a result, the internal lens 1230 lowers overall lens power as well as minimizes chromatic aberration of the lens. The interior chamber 1215 may be filled with an optical fluid having an Abbe number different from that of the internal lens element 1230 so that Eq. 1 is satisfied. In some embodiments, the Abbe number of the shell material is used to decrease chromatic aberration. This includes using materials having a high Abbe number—generally above 20, in some embodiments above 40. Typically, the Abbe number ranges from 20 to 100.

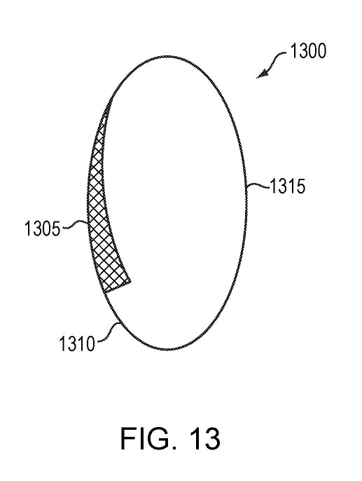

In still other embodiments, a diffractive optic (such as a diffractive lens or a diffractive diffuser) is placed on or integrated with the lens to reduce chromatic aberration. For example, as shown in FIG. 13, an IOL 1300 includes a diffractive optic 1305 disposed on the anterior portion 1310 (and/or the posterior portion 1315) of the lens shell. The diffractive element 1315 may be on the inside or outside of the shell, or may extend through the shell. The diffractive optic 1305 reduces chromatic aberration of the lens.

Examples of filling fluids that can be used in the lens embodiments discussed herein include, but are not limited to, silicone oils, modified silicone oils or gels such as phenyl-substituted oils/gels, fluorosilicones, perfluorocarbons, aqueous solutions such as glucose or dextrose and water, curable gels, curable polymers, and hydrogels. Fluids may include dispersions of small particles, such as nanoparticles, microbubbles, nanobubbles, etc. In certain embodiments, the nanoparticles alter optical properties such as refraction and/or transmission as well as dispersive, mechanical, and viscous properties. Examples of transmission properties include ultraviolet light-blocking properties, color-altering properties, or photochromic properties.

As noted, IOLs in accordance herewith may include one or more refill valves. Refill valves may be self-sealing and selected (in terms of size, thickness and flexibility) to avoid affecting the characteristics of the optical zone (e.g., by creating additional optical aberrations). In embodiments containing regions of different transmissivity, different optical material, different optical fluids, etc., the valve(s) may be index-matched to the region, e.g., to exhibit the same or neutral optical qualities so as not to optically affect the region. The refill valves may be accessed on multiple occasions: prior to insertion to remove fluid in order to reduce the overall conformation for insertion through a small (e.g., less than 3 mm) incision, during implantation to inflate the IOL to fit the capsular bag, post-implantation to adjust for the effect of healing fibrosis on the size of the capsular bag, unwanted aberrations, and accommodation-related DOF to be tailored for each individual patient.

Shell materials include but are not limited to acrylics, silicones, fluorosilicones, phenyl silicones, parylene, composite and blended materials (e.g., silicone/parylene), and nanocomposites. Nanoparticles may be included the shell material to cause a gradient index of the material. In other embodiments they are used to alter optical properties such as refractive properties, transmission properties as well as dispersive properties of the fluid, mechanical properties, and viscous properties. It should also be understood that the shell of an IOL may vary in thickness or composition in order to both provide the necessary corrective optics, accommodation response characteristics, and to provide a difference in Abbe number from the internal filling fluid in order to satisfy Eq. 1.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An intraocular lens comprising a membrane defining a central chamber for containing an optical fluid and, when filled, to provide vision correction when implanted in a patient's eye, the membrane having an optical axis and opposed anterior and posterior optical surfaces perpendicular to the optical axis, wherein a first portion of at least one of the optical surfaces has a higher optical density than a second portion thereof different from the first portion, such that more light passes through the second portion than the first portion.

2. The intraocular lens of claim 1, wherein the second portion has a circular shape and is centrally located on the corresponding optical surface.

3. The intraocular lens of claim 2, wherein the central portion has a diameter of 3 mm.

4. The intraocular lens of claim 2, wherein the first portion is a peripheral portion surrounding the second portion.

5. The intraocular lens of claim 4, wherein the first portion has an absorbance of at least 70% for visible light.

6. The intraocular lens of claim 4, wherein the first portion has an absorbance of at least 95% for visible light.

7. The intraocular lens of claim 2, wherein the peripheral portion of higher optical density is on the anterior surface.

8. The intraocular lens of claim 2, wherein the peripheral portion of higher optical density is on the posterior surface.

9. The intraocular lens of claim 2, wherein the peripheral portion comprises a plurality of concentric zones of differing optical densities.

10. The intraocular lens of claim 9, wherein the concentric zones increase in optical density with radial distance from the central zone.

11. The intraocular lens of claim 2, wherein the peripheral portion increases continuously in optical density from the central portion.

12. The intraocular lens of claim 11, wherein the peripheral portion increases linearly in optical density from the central portion.

13. The intraocular lens of claim 11, wherein the peripheral portion increases nonlinearly in optical density from the central portion.

14. The intraocular lens of claim 1, wherein the second portion is a pattern of apertures distributed over corresponding optical surface.

15. The intraocular lens of claim 1, wherein at least one of the anterior and posterior optical surfaces comprises:
a first, central lens portion having a first optical power; and
a second lens portion at least partially surrounding the first lens portion, the second lens portion having a second optical power different from the first optical power.

16. The intraocular lens of claim 15, wherein the second lens portion is a peripheral portion discrete from and fully surrounding the first lens portion.

17. The intraocular lens of claim 16, wherein the second lens portion is annular.

18. The intraocular lens of claim 15, further comprising a third lens portion, the second and third lens portions being discrete, having different lens powers, and collectively surrounding the first lens portion.

19. The intraocular lens of claim 1, wherein at least one said optical surface has an Abbe number different from each other and/or from the optical fluid.

20. The intraocular lens of claim 1, further comprising a diffractive element on at least one of the anterior or posterior optical surfaces.
